(19) United States
(12) Patent Application Publication
(10) Pub. No.: US 2013/0211270 A1
(43) Pub. Date: Aug. 15, 2013

(54) Mouth Guard for Monitoring Body Dynamics and Methods Therefor

(71) Applicants: Bryan St. Laurent, Atlanta, GA (US); Gregory D. Durgin, Atlanta, GA (US); Matthew Shayaun Trotter, Pittsburgh, PA (US)

(72) Inventors: Bryan St. Laurent, Atlanta, GA (US); Gregory D. Durgin, Atlanta, GA (US); Matthew Shayaun Trotter, Pittsburgh, PA (US)

(21) Appl. No.: 13/588,663

(22) Filed: Aug. 17, 2012

Related U.S. Application Data
(63) Continuation-in-part of application No. 12/505,916, filed on Jul. 20, 2009.
(60) Provisional application No. 61/525,001, filed on Aug. 18, 2011.

(51) Int. Cl. A61B 5/00; A61B 5/024; A61B 5/08; A61B 5/01

(52) U.S. Cl. CPC A61B 5/682; A61B 5/01; A61B 5/024; A61B 5/08; A61B 5/4875
USPC 600/508; 600/300; 600/595; 600/549; 600/529; 600/553

(57) ABSTRACT

An electronic monitoring device (100) is configured as a mouth guard (101). The mouth guard includes a biometric sensor (106) and a communication device (107). The communication module includes an antenna (406), a controller (404); and a switch (408) operative to change a radar cross section of the antenna by selectively altering a load impedance of the antenna. The controller backscatters received radio frequency signals (415) by modulating the received radio frequency signal by controlling the switch to encode output from the biometric sensor therein. In a method (800), a coach, trainer, or parent can monitor biometric activity of a user by receiving backscattered return signals having output of the biometric sensor encoded therein. Detection of location, other biometric information, and user identification is also possible.

### CROSS REFERENCE TO PRIOR APPLICATIONS

This application claims priority and benefit under 35 U.S.C. §119(e) from U.S. Provisional Application No. 61/525,001, filed Aug. 18, 2011, which is incorporated by reference for all purposes. This application is a continuation-in-part of U.S. application Ser. No. 12/505,916, filed Jul. 20, 2009, which is incorporated herein by reference for all purposes, and which claims priority and benefit under 35 U.S.C. §119(e) from U.S. Provisional Application No. 61/083,974, filed Jul. 28, 2008.

### BACKGROUND

1. Technical Field

This invention relates generally to sensors, and more particularly to biometric sensors.

### BACKGROUND ART

Sporting enthusiasts and athletes play, practice, and train for the sports in which they participate. It would be beneficial to have devices and methods to more effectively monitor their activities.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one embodiment of an electronic monitoring device configured in accordance with one or more embodiments of the invention.

FIG. 2 illustrates another embodiment of an electronic monitoring device configured in accordance with one or more embodiments of the invention.

FIG. 3 illustrates yet another embodiment of an electronic monitoring device configured in accordance with one or more embodiments of the invention.

FIG. 4 illustrates an explanatory reflected interferometry system suitable for communication with an electronic monitoring device configured in accordance with one or more embodiments of the invention.

FIG. 5 illustrates yet another embodiment of an electronic monitoring device configured in accordance with one or more embodiments of the invention.

FIG. 6 illustrates yet another embodiment of an electronic monitoring device configured in accordance with one or more embodiments of the invention.

FIG. 7 illustrates one explanatory biometric monitoring system configured in accordance with one or more embodiments of the invention.

FIG. 8 illustrates one explanatory method of monitoring anatomical motion configured in accordance with one or more embodiments of the invention.

FIG. 9 illustrates another explanatory reflected interferometry system suitable for communication with an electronic monitoring device configured in accordance with one or more embodiments of the invention.

FIG. 10 illustrates one explanatory informational display from one biometric monitoring system configured in accordance with one or more embodiments of the invention.

FIG. 11 illustrates an identification determination system suitable for use in a biometric monitoring system configured in accordance with one or more embodiments of the invention.

FIG. 12 illustrates another explanatory biometric monitoring system configured in accordance with one or more embodiments of the invention.

FIG. 13 illustrates explanatory waveforms from one or more reflected interferometry communication devices configured in accordance with one or more embodiments of the invention.

FIG. 14 illustrates one explanatory method of monitoring anatomical motion in accordance with one or more embodiments of the invention.

FIG. 15 illustrates another method for monitoring anatomical motion in accordance with one or more embodiments of the invention.

FIG. 16 illustrates a location determination method suitable for use with one or more biometric monitoring systems configured in accordance with one or more embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF EMBODIMENTS OF THE INVENTION

Before describing in detail embodiments that are in accordance with the present invention, it should be observed that the embodiments reside primarily in combinations of method steps and apparatus components related to biometric monitoring with an electronic monitoring device employing reflected interferometry communication. Any process descriptions or blocks in flow charts should be understood as representing modules, segments, or portions of code which include one or more executable instructions for implementing specific logical functions or steps in the process. Alternate implementations are included, and it will be clear that functions may be executed out of order from that shown or discussed, including substantially concurrently or in reverse order, depending on the functionality involved. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

It will be appreciated that embodiments of the invention described herein may be comprised of one or more conventional processors and unique stored program instructions that control the one or more processors to implement, in conjunction with certain non-processor circuits, some, most, or all of the functions of electronic biometric monitoring described herein. The non-processor circuits may include, but are not limited to, a radio receiver, a radio transmitter, signal drivers, clock circuits, power source circuits, and user input devices. As such, these functions may be interpreted as steps of a method to perform biometric monitoring. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the present invention provide an electronic monitoring device that includes one or more biometric sensors and a communication device. In one embodiment, the electronic components are coupled to, or alternatively integrated in, a mouth guard. Examples of biometric sensors include force sensors and temperature sensors. Electronic monitoring devices configured in accordance with embodiments of the invention are configured to provide biometric data to a receiver in real time. Through the use of reflected interferometry communication, embodiments described herein provide advantages over prior art solutions in that large power supplies and high-powered radio transceivers need not be incorporated into the monitoring device. This advantage allows the mouth guard to be smaller and lighter, more resembling traditional mouth guards worn by athletes.

In one embodiment, the electronic monitoring device is configured as a mouth guard suitable for protecting an athlete's teeth during sporting activities. One or more biometric sensors can then be attached to or integrated with the mouth guard. A communication module, operable with the biometric sensor, then includes an antenna, a controller and a switch. The switch is responsive to the controller and is operative to change a radar cross section of the antenna by selectively altering a load impedance of the antenna. The controller is configured to receive biometric data and/or signals from the biometric sensor. The controller then, upon receiving radio frequency signals from a transceiver, is configured to backscatter received radio frequency signals by modulating the received radio frequency signal by controlling the switch to encode output from the biometric sensor into a backscattered return signal.

When configured as a system, the electronic monitoring device can be used with one or more remote radio frequency transceivers. A monitoring module is operable with the one or more radio frequency transceivers. One or more monitoring devices can be given, for example, to one or more athletes competing in an activity on an athletic field. In one embodiment, the one or more radio frequency transceivers are each configured to transmit a radio frequency signal. They then receive a backscattered return signal from the each of the monitoring devices. In one embodiment, the backscattered return signals comprise multi-path, and frequently indirect path, return signals. The backscattered return signals in one embodiment have an output from the biometric sensor modulated therein due to the controller of a particular monitoring device switching the switch. In another embodiment, the backscattered return signals also include a unique identifier so the identity of a particular athlete can be determined when multiple monitoring devices are in use. Further, when multiple radio transceivers are in use, embodiments of the invention can determine the location of each monitoring device in real time as well.

Embodiments of the invention accordingly provide a method of monitoring anatomical motion and activity. In one embodiment, this includes providing one or more users with mouth guards. Each mouth guard can include a biometric sensor and a switch operable to change a radar cross section of an antenna of the mouth guard by selectively altering a load impedance of the antenna. A coach, parent, or other person can then monitoring biometric activity of the user by transmitting, with one or more transceivers, a radio frequency signal and receiving backscattered return signals having output of the biometric sensor encoded therein. This method can be used to monitor for critical biometric events, including potential overheating, dehydration, and concussions or trauma due to impact, just to name a few.

Embodiments of the invention contemplate that monitoring head motion and vital signs in physical activities provides valuable information that can lead to the prevention of medical problems during the physical activity and in the future. The electronic monitoring devices configured in accordance with the description below allow monitoring, in one embodiment, of head motion dynamics such as impulsive and translational force in three dimensions, impulsive and smooth rotational forces in two dimensions, and head orientation in three dimensions. In addition, some embodiments of the electronic monitoring devices described below can be used to monitor other vital signs such as ambient mouth temperature, heart rate, and mouth moisture. Embodiments of the invention are suitable for monitoring health diagnostics data, taking scientific measurements, and recording statistics for sports and physical activities.

Embodiments of the invention are especially useful in monitoring forceful impacts to the head that may occur in sporting activities. Recent studies suggest that measuring impacts to the head with linear accelerations in excess of 96.1 G's or rotational accelerations in excess of 5582.3 rad/s2 correlate with concussion incidences. Accordingly, embodiments of the invention can be used as concussion monitoring devices in high-impact sports such as football and rugby. In such sports, mouth guards are standard equipment. By configuring the electronic monitoring device as a mouth guard, the athlete can be monitored without altering standard equipment. Such monitoring is beneficial in sports with head impacts such as football, hockey, rugby, soccer, boxing, and mixed martial arts. The measurement of head acceleration can be used as a diagnostic for detecting concussions, muscle strain, neck injuries, and internal bruises. Medical procedures for diagnosing concussions are performed immediately after a severe blow to the head or neck. Symptoms such as headache, dizziness, nausea, or loss of consciousness typically indicate a concussion. Further testing such as x-ray, CT scan, or neurological testing can diagnose a concussion. The data from embodiments of the invention are unique in that it is measured data of the hit instead of a symptom of the hit.

A secondary use of embodiments of the invention is as an accurate scientific measurement tool. Embodiments can be used to scientifically measure linear or rotational acceleration and biometric data. Embodiments described below can feasibly contain any number of biometric sensors that measure vital signs such as heart rate, temperature, moisture level, or blood pressure. In addition, any biometric data that can be measured using a pair of electrodes connected to the roof of the mouth, the tongue, or teeth can be measured by providing one or more exposed electrodes along the surface of the mouth guard.

Another use for embodiments of the invention includes simply recording biometric data during physical activity. Embodiments of the invention can track and store biometric data over the course of the physical activity for later reporting. Coaches, trainers, and athletes can use the information for optimizing physical performance or in any other optimization.

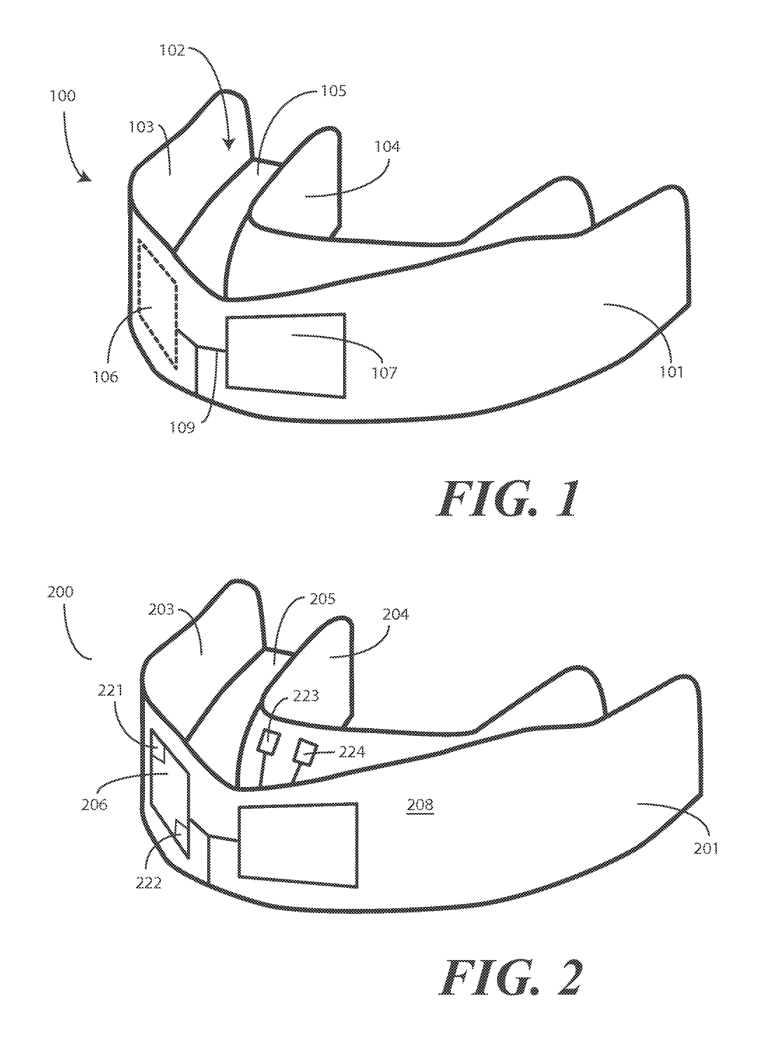

Turning now to FIG. 1, illustrated therein is one embodiment of an electronic monitoring device 100 configured in accordance with one or more embodiments of the invention. The electronic monitoring device 100 includes a mouth guard 101, which may be formed from a rubberized plastic or thermoplastic material. The mouth guard 101 defines a channel 102 having a first wall 103 on one side of the channel 102 and a second wall 104 on the opposite side of the channel 102. The channel is configured to receive a user's teeth when the mouth guard 101 is worn. In this illustrative embodiment, the channel 102 is open on the top and bounded by a floor 105 on the bottom.

The mouth guard 101 has coupled thereto—integrated therein—a biometric sensor 106 and a communication device 107. In this illustrative embodiment, the biometric sensor 106 is integrated into the body of the mouth guard 101, while the communication device 107 is disposed along a surface 108 of the mouth guard 101. In other embodiments, both the biometric sensor 106 and the communication device 107 can be disposed along the surface 108 of the mouth guard 101. In still other embodiments, the biometric sensor 106 and the communication device 107 can be integrated into the body of the mouth guard 101. Note that while a single biometric sensor 106 is shown in FIG. 1 for simplicity, as will be described below, embodiments of the invention can be configured with two, three, four, or more biometric sensors.

The biometric sensor 106 can be configured as any of a number of different types of sensors. For example, in one embodiment, the biometric sensor comprises a linear acceleration detector. In another embodiment, the biometric sensor 106 comprises a rotational acceleration sensor. In another embodiment, the biometric sensor 106 comprises both a linear acceleration detector and a rotational acceleration sensor. In another embodiment, the biometric sensor 106 comprises a temperature sensor. In another embodiment, the biometric sensor 106 comprises a moisture sensor. In another embodiment, the biometric sensor 106 comprises a heart rate sensor. In another embodiment, the biometric sensor 106 comprises a respiration sensor. Of course, combinations of these sensors can be used as well. Moreover, those of ordinary skill in the art will recognize that other types of biometric sensors can be used instead of, or in addition to, the sensors listed above. Accordingly, the list is intended to be illustrative only, and is not limiting. For example, other types of biometric sensors include microelectromechanical (MEMS) type impact sensors, MEMS accelerometers, and/or miniature weighted cantilevers fitted with miniature strain-gauge elements, piezoelectric membranes, or force-sensitive resistors.

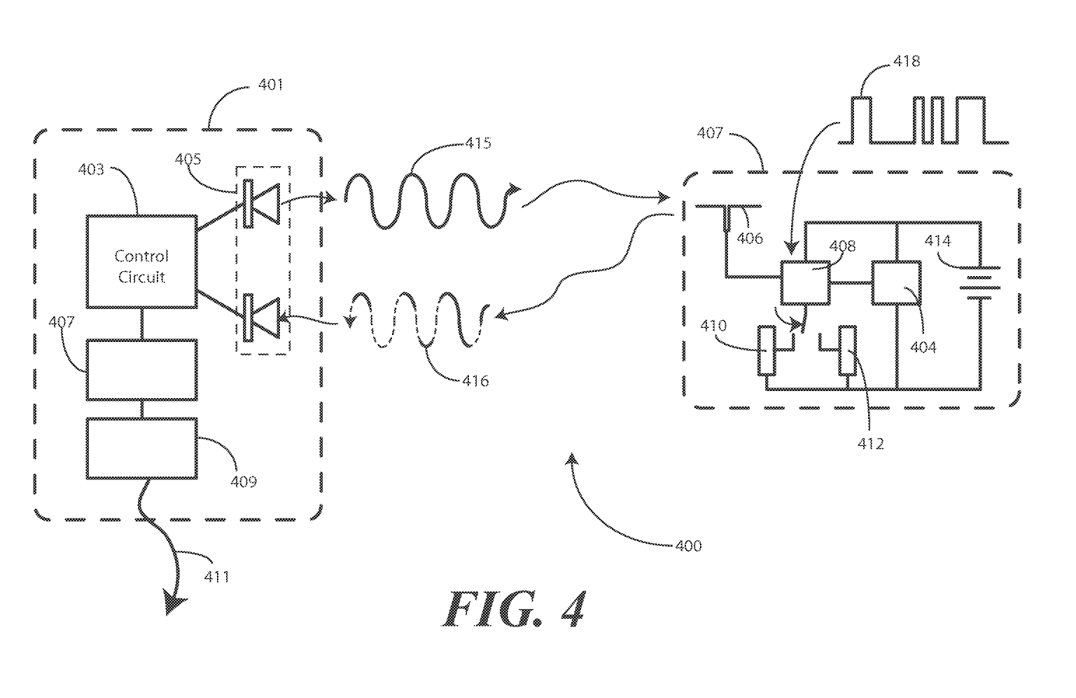

In one embodiment, the biometric sensor 106 comprises a linear acceleration sensor, a rotational acceleration sensor, or a combination thereof. The communication device 107 is operable with the biometric sensor and receives sensed acceleration data from the biometric sensor via a communication bus 109. A radio frequency transceiver, which will be explained in more detail with reference to FIG. 4 below, located near a user wearing the mouth guard 101 is used to communicate wirelessly with the communication device 107 and to receive acceleration data encoded in return radio frequency communications from the electronic monitoring device 100. By using a plurality of radio frequency transceivers, a large area, e.g., a football field, can be covered and multiple electronic monitoring devices 100 can be used concurrently to monitor multiple users.

In one embodiment, the acceleration detectors of the biometric sensor 106 are configured to monitor linear acceleration and/or rotational acceleration in three Cartesian dimensions. This monitoring is used in one embodiment to monitor for concussions in athletes. With a Cartesian plane being defined by X, Y, and Z coordinates, linear acceleration of the head of a user who has the mouth guard 101 in his mouth can be measured in the x, y, and z axes. Rotational acceleration around the x, y, and z axes can also be measured using a convention corresponding to the “right-hand rule”. As is known in the art, the right hand rule defines a convention when one points the right-hand thumb in the positive linear direction of the axis, and then wraps the right-hand fingers around the axis in the positive direction of rotation for that axis.

When force is applied to a user wearing the mouth guard 101, the measured linear and rotational acceleration vectors are represented as:

A.sub.lin=a.sub.x*x.sub.vect+a.sub.y*y.sub.vect+a.sub.z*z.sub.vect; (EQ. 1)

and

A.sub.rot=a.sub.x*Omega.sub.x+a.sub.y*Omega.sub.y+a.sub.z*Omega.sub.z; (EQ. 2)

where x.sub.vect, y.sub.vect, and z.sub.vect are the linear basis vectors that point in the positive x, y, and z directions respectively, and the rotational basis vectors Omega.sub.y, Omega.sub.y, and Omega.sub.z point in the positive rotation directions around the x, y, and z axes respectively. The amount of “head jerk” resulting from an impact force can be measured as well by taking the derivative of measured acceleration versus time as follows:

Jerk.sub.lin=d/dt(A.sub.lin); and (EQ. 3)

Jerk.sub.rot=d/dt(A.sub.rot). (EQ. 4)

Significant magnitudes of head jerk can correspond to probable incidence of concussion. For example, a sudden hit to the head between the left eye and left ear may cause both linear and rotational acceleration. Significant linear acceleration would be measured in the negative y and z directions, and significant rotational acceleration would be measured around the z axis. The biometric sensor 106, in one embodiment, detects this acceleration, and delivers signals corresponding thereto to the communication device 107. The communication module then delivers this data to radio frequency transceivers by backscattering return signals having output of the biometric sensor encoded therein. When other sensors are included in the biometric sensor 106, other data can be reported in the same way, including but not limited to, heart rate, moisture content, and mouth temperature.

While acceleration can be measured with a biometric sensor 106 that is integrated into a body of the mouth guard 101, some other information requires exposed measurement terminals. Turning now to FIG. 2, illustrated therein is another electronic monitoring device 200 that is equipped with such terminals. Specifically, in FIG. 2, the biometric sensor 206 includes not only acceleration sensors, but also a temperature sensor 221 and a moisture sensor 222. The temperature sensor 221 can be used to monitor biometric activity and warn when overheating may occur. The moisture sensor 222 can be used to monitor for dehydration.

To enable more efficient temperature sensing, in one embodiment the temperature sensor 221 is operative with a first biometric sensor terminal 223 that has a surface exposed to a surface 208 of the mouth guard 201. When the mouth guard 201 is placed in the mouth of a user, the first biometric sensor terminal 223 can rest against the roof of the mouth in this illustrative embodiment to better detect temperature. Similarly, in one embodiment the moisture sensor 22 is operative with a second biometric sensor terminal 224 that has one or more surfaces exposed to the surface 208 of the mouth guard 201. The second biometric sensor terminal 224 can thus sense moisture in the mouth. Note that while the first biometric sensor terminal 223 and the second biometric sensor terminal 224 are disposed on an upper surface in this illustrative embodiment, it will be clear to those of ordinary skill in the art having the benefit of this disclosure that other configurations could also be used. For example, one or both of the first biometric sensor terminal 223 and the second biometric sensor terminal 224 could be disposed on a first sidewall 203 of the mouth guard 201, a second sidewall 204 of the mouth guard 201, or on a floor 205 of the mouth guard 201 as well.

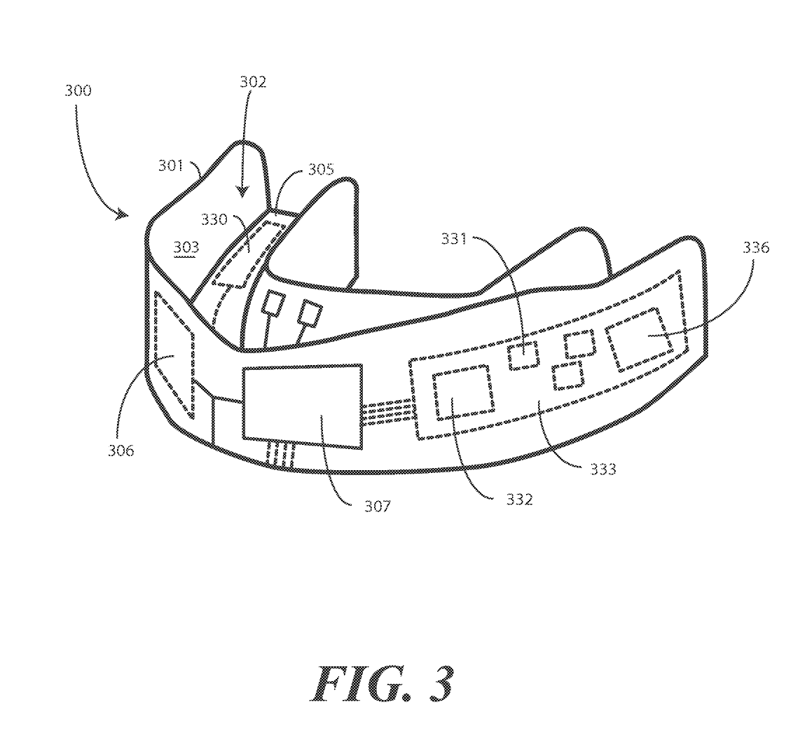

Turning now to FIG. 3, illustrated therein is another embodiment of an electronic monitoring device 300 configured in accordance with one or more embodiments of the invention. In this illustrative embodiment, the mouth guard 301 includes as a power source a generator 330. The generator 330 can provide power to the various electrical components of the electronic monitoring device 300 by creating power in response to biometric activity of a wearer. For example, in this illustrative embodiment, the generator 330 comprises a piezoelectric generator disposed along a floor 305 of the mouth guard 301. When the user clenches their teeth about the floor 305, pressure is exerted on the generator 330. The generator 330 converts this pressure into electricity to power the various electronic components. As will be described below with reference to FIG. 4, in one embodiment the communication device 307 comprises a backscattering interferometry device which uses vary small amounts of power. Such devices are well suited for compact generators such as piezoelectric devices due to their low power consumption.

In other embodiments, the generator 330 can be replaced by a battery. Such a battery can be coupled to the biometric sensors 306,336 and the communication device 307. In some embodiments, the batteries are rechargeable, such as via a wireless charging device. However, due to the low power consumption of the communication device 307, in other embodiments the battery will be a primary use battery. When the primary use battery is depleted, the electronic monitoring device 300 will simply be discarded.

In yet other embodiments, the generator 330 can be replaced with a power harvester. As will be described below, in one or more embodiments the communication device 307 communicates by backscattering received radio frequency waves. The power harvester allows the electronic monitoring device 300 to function without batteries. One example of a suitable power harvester is described in D. Dobkin's book, “The RF in RFID. Passive UHF RFID in Practice,” published by Elsevier in 2008. Using the power harvester, power for the biometric sensors 306,336 and the communication device 307 can be harvested from an un-modulated signal received at an antenna of the communication device 307.

In this illustrative embodiment, one biometric sensor 306 is disposed along a front portion of the mouth guard 301. This biometric sensor 306, in this illustrative embodiment, would be proximately located with the incisors of a user when the mouth guard 301 is placed in the user's mouth with the user's upper teeth disposed in the channel 302. Biometric sensor 336 is disposed at a different area of the mouth guard 301.

In this illustrative embodiment, biometric sensor 306 comprises a three-axis accelerometer configured to detect acceleration along three orthogonal linear axes. Operable with biometric sensor 306 is a three-axis gyroscope 331 and a control circuit 332 that are disposed on a flexible substrate 333 that is integrated into the mouth guard 301. The three-axis accelerometer, in one embodiment continually monitors acceleration and outputs an impact-warning signal when acceleration is measured in excess of 90 G. In one embodiment, the three-axis gyroscope 331 is configured to resolve six thousand or more degrees per second. In one embodiment, each of biometric sensor 306, the communication device 307, and the flexible substrate 333 are disposed on the first sidewall 303, which is outside the user's teeth. This helps to ensure that the user's teeth do not interfere with backscattering signals from the communication device 307.

As described above, the three-axis accelerometer and gyroscope 331 can be configured to determine a rate of acceleration of the mouth guard 301. Additionally, these devices can determine orientation of the mouth guard 301 in time. By correlating acceleration and position, the electronic monitoring device can not only the fact of an impact of a particular magnitude has occurred, but also the direction of the impact from the direction of movement of the gyroscope 331. These data can be used to calculate a vector representative of a combined direction and magnitude of the acceleration experienced by the electronic monitoring device 300. In some instances the calculated vector may be along a straight line, while in other instances the calculated vector may be curvilinear, rotational, or combinations thereof.

While a three-axis accelerometer is one explanatory biometric sensor 306, it will be clear to those of ordinary skill in the art that embodiments of the invention are not so limited. For example, in another embodiment, biometric sensor 306 comprises three linear accelerometers. Moreover, gyroscope 331 may not be required.

Turning now to FIG. 4, illustrated therein is a schematic block diagram of a communication device 107 in operation with a radio frequency transceiver 401. FIG. 4 illustrates how communication devices 107 (207,307) backscatter received radio frequency signals in accordance with one or more embodiments of the invention.

In the illustrative embodiment of FIG. 4, a radio transceiver 401 and a communication device 107 suitable for coupling to or integration in a mouth guard (101) in accordance with one or more embodiments of the invention are shown. The illustrative radio transceiver 401 includes a control circuit 403 and a transceiver 405. The illustrative communication device 107 includes a controller 404, an antenna 406, and a switch 408 that is responsive to the controller 404. The switch 408 is operative to change a radar cross section of the antenna 406 by selectively altering a load impedance of the antenna 406. In the illustrative embodiment of FIG. 4, the switch 408 does this by selectively switching between two loads 410,412 coupled to the antenna 406. The first load 410 is a high impedance, so as to resemble a substantially open circuit, while the second load 412 is a low impedance configured to resemble a substantially short circuit. An on-board battery 414, such as a small lithium-ion or lithium-polymer battery suitable for use in wristwatches, provides power for the controller 404 and the switch 408. As noted above, the battery 414 can be replaced either by a generator or power harvester, each of which is alternatively represented by dashed box (330) of FIG. 3.

Examples of the controller 404 disposed in the communication device 107 and control circuit 403 coupled to the transceiver 405 include a microprocessor configured to execute instructions stored in an on-board or separately coupled memory. Alternatively, each of the controller 404 and control circuit 403 can be configured as programmable logic, an ASIC, or combinations thereof. The radio transceiver 401 can even be configured such that the control circuit 403 is disposed in a portable computer or other electronic control device that is electrically coupled with the transceiver 405. In at least some instances, the controller 404 and control circuit 403 will be implemented using one or more microprocessors, implemented to execute one or more sets of pre-stored instructions. However in some instances all or portions of the controller could be implemented in hardware, where exemplary forms include one or more sequential state machines and/or various logic circuitry, including discrete logic elements, programmable gate array elements, or VLSI circuitry. It will be obvious to those of ordinary skill in the art having the benefit of this disclosure that other alternative implementations involving various forms of software programming and hardware elements can be used to implement embodiments of the present invention without departing from the teachings herein.

The transceiver 405 of the radio transceiver 401 is responsive to the control circuit 403 and is configured to transmit a radio frequency signal 415. Where multiple radio transceivers are used in an application, the radio frequency signal 415 transmitted by each radio transceiver may be unique. For example, in one embodiment, each radio frequency signal 415 transmitted may be offset from the others by a predetermined phase. In another embodiment, each radio frequency signal 415 transmitted may have a different frequency or characteristic waveform. In one embodiment, the transmitted radio frequency signal 415 has a center frequency of about 915 MHz, about 2.45 GHz, or about 5.7 GHz. These frequencies are well suited to embodiments of the invention in that they represent the unlicensed scientific and medical bands of 915 MHz, 2.45 GHz, and 5.7 GHz, respectively, having wavelengths of 30 centimeters, 12 centimeters, and 5 centimeters respectively. It will be clear to those of ordinary skill in the art having the benefit of this disclosure, however, that embodiments of the present invention are not limited to these frequencies, as any number of radio frequency bands may work as well.

The communication device 107 receives this radio frequency signal 415 at its antenna 406. In one embodiment, the antenna 406 comprises a slot antenna suitable for receiving radio frequency communication. In one embodiment, the antenna 406 comprises an inverted-F antenna.

The switch 408 of the communication device 107 then, in response to a control signal from the controller 404, switches in accordance with biometric data signals 418 received from the biometric sensor (106). In one embodiment, the switch 408 switches between the two loads 410,412, thereby changing the radar cross-section of the antenna 406. This change in radar cross section serves to modulate or encode the biometric data signals 418 into a backscattered return signal 416 that is backscattered from the antenna 406. The transceiver 405 then receives this backscattered return signal 416 having the biometric data modulated therein. By reading the biometric data, the control circuit 403 is able to determine biometric information about the user wearing the electronic monitoring device.

A biometric evaluation device 407 then processes the biometric information. A modeling device 409 can then receive information from the biometric evaluation device 407 to create diagnostic models from the data. Illustrating by example, the modeling device 409 can translate linear and/or rotational forces from the biometric data signals 418 to a center of mass of an athlete's head. The modeling device 409 can then deliver 411 to a display so that a coach, parent, or medical professional can view a graphical representation of the linear and/or rotational forces on the athlete's head. In some embodiments, the coach, parent, or medical professional can also see graphical representations of the athlete's temperature, oral moisture, heart rate, or other vital signals. In one or more embodiments, the biometric evaluation device 407 includes an injury warning device. The injury warning device can generate alerts when the biometric data signals correspond to thresholds representative of injury to the athlete. In one embodiment, the injury warning device is connected across a network like the Internet to a medical record system to draw data corresponding to injuries.

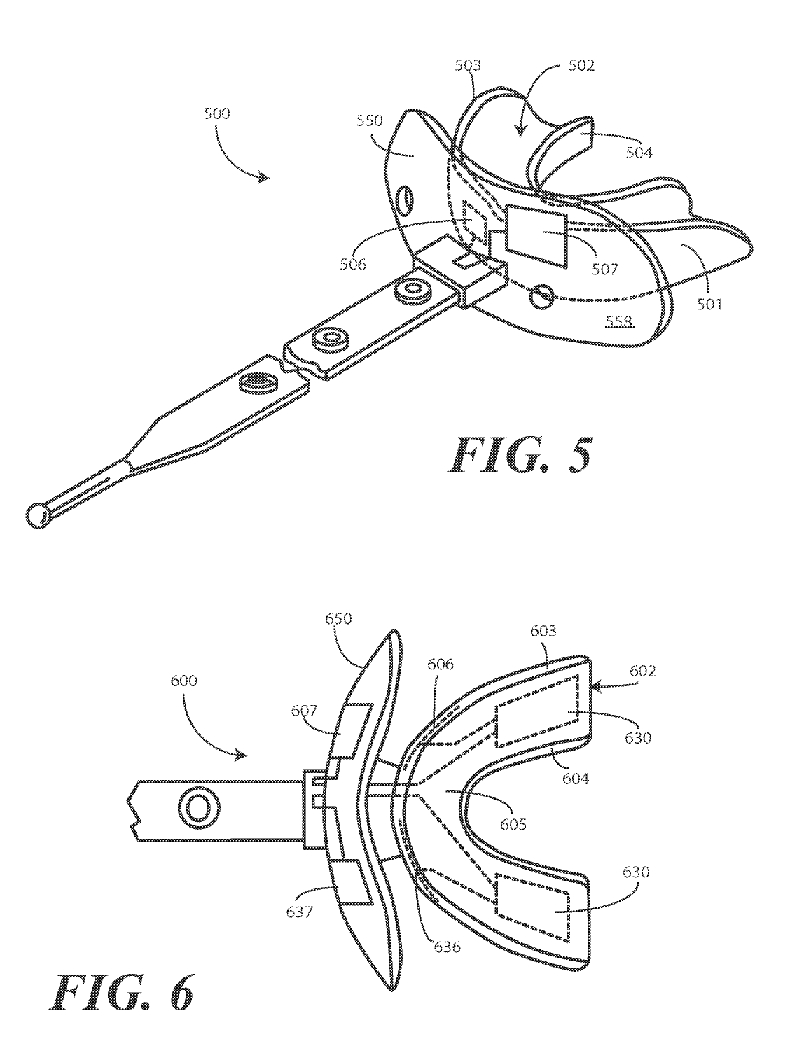

Turning now to FIG. 5, illustrated therein is an alternate electronic monitoring device 500 configured in accordance with one or more embodiments of the invention. As with previous embodiments, the electronic monitoring device 500 of FIG. 5 includes a mouth guard 501, which may be formed from a rubberized plastic or thermoplastic material. The mouth guard 501 defines a channel 502 having a first wall 503 on one side of the channel 502 and a second wall 504 on the opposite side of the channel 502. The channel is configured to receive a user's teeth when the mouth guard 501 is worn. The mouth guard 501 also has coupled thereto—integrated therein—a biometric sensor 506 and a communication device 507.

In addition to having a mouth guard 501, the explanatory electronic monitoring device 500 of FIG. 5 has a lip guard 550 as well. Specifically, the mouth guard 501 defines a channel 551 between the first wall 503 and the lip guard 550 that is configured to receive a user's lips. As it will be appreciated that a user's lips can degrade any radio frequency signal received by the communication device 507, or any backscattered signals therefrom. The embodiment of FIG. 5 precludes this degradation by placing the biometric sensor 506 in the mouth while the communication device 507 is disposed outside the mouth on the lip guard 550. Said differently, in this explanatory embodiment the biometric device 506 is disposed on a first side of the lip channel 551 while the communication device 507 is disposed on a second side of the lip channel 551. In this illustrative embodiment, the biometric sensor 506 is integrated into the body of the mouth guard 501, while the communication device 507 is disposed along a surface 558 of the mouth guard 501.

Turning now to FIG. 6, illustrated therein is yet another electronic monitoring device 600 configured in accordance with embodiments of the invention. The electronic monitoring device 600 of FIG. 6 is similar to that shown in FIG. 5, in that in addition to having a mouth guard 601, the explanatory electronic monitoring device 600 of FIG. 6 has a lip guard 650 as well. In this illustrative embodiment, two communication devices 607,637 are disposed on the lip guard 650 while two biometric sensors 606,636 are disposed on the mouth guard 601. Further, two generators 630 are disposed along the floor 605 of the mouth guard 501.

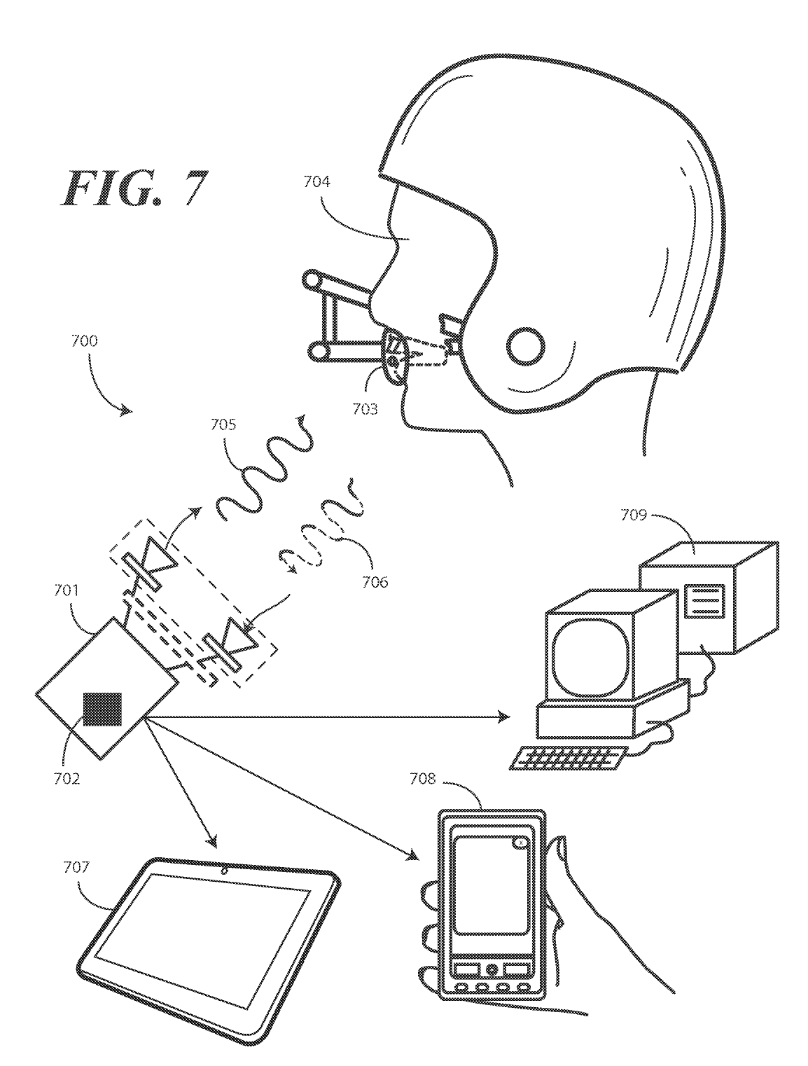

Turning now to FIG. 7, illustrated therein is one embodiment of a biometric monitoring system 700 configured in accordance with embodiments of the invention. The biometric monitoring system 700 includes a radio frequency transceiver 701, a monitoring module 702 that is operable with the radio frequency transceiver 701, and a monitoring device 703, shown here as a mouth guard inserted into the mouth of an athlete 704. The athlete 704 of this illustrative embodiment is a football player. However, as noted above, the athlete 704 could be competing in other sports as well. Also, while one radio frequency transceiver 701 is shown for simplicity, it should be understood that radio frequency transceiver 701 could represent a plurality of radio frequency transceivers as well.

As with previous embodiments, the monitoring device 703 comprises a biometric sensor and a communication module. The communication module is operable with the biometric sensor and includes an antenna, a controller, and a switch. The switch is responsive to the controller. The switch is operative to change a radar cross section of the antenna by selectively altering a load impedance of the antenna.

As shown in FIG. 7, the radio frequency transceiver 701 is configured to transmit a radio frequency signal 705. The radio frequency signal 705 is received by the communication module of the monitoring device 703. The switch of the communication module then changes a radar cross section of the antenna of the communication module in accordance with output signals from the biometric sensor to modulate information from the biometric sensor into a return signal 706. The return signal is then backscattered from the monitoring device 703 to the radio frequency transceiver 701. In one embodiment, this backscattering comprises delivering multipath signals back to the radio frequency transceiver 701. In one embodiment, these multipath signals are scattered in that they travel non-linear, multidirectional paths back to the radio frequency transceiver 701. The radio frequency transceiver 701 thus receives a backscattered return signal from the monitoring devices 703, with the backscattered return signal having an output from the biometric sensor modulated therein due to the controller switching the switch.

The monitoring module 702 then processes the received data and delivers it, wired or wirelessly, to one or more monitoring devices. Said differently, the monitoring module 702 is configured to collect the output from the biometric sensor, received by the radio frequency transceiver 701 as a backscattered return signal, and present indicia corresponding thereto on a display of a monitoring device. In one embodiment, the indicia comprise data corresponding to an impact event detected by the biometric sensor of the monitoring device 703. The indicia can further comprise a concussion warning. The monitoring devices can include a tablet 707, a mobile device 708, or a computer 709. Other monitoring devices can be used as well, as will be obvious to those of ordinary skill in the art having the benefit of this disclosure.

In one or more embodiments, the biometric monitoring system 700 can be used to aggregate head-acceleration information received from the monitoring device 703. To facilitate ease of monitoring, the monitoring module 702 of the radio frequency transceiver can be in wireless communication with one or more of the monitoring devices to allow coaches, parents, and/or spectators to monitor not only monitor head acceleration of the various players, but temperature information, hydration information, body temperature information, heart rate information, and other vital information for each player. In one or more embodiments, the monitoring module 702 is operable with a server, network, or other electronic device that serves as an intermediate device between the radio frequency transceiver and the monitoring devices. Additional processing capabilities can be integrated into the server or other electronic device as well. The monitoring device can include its own processor, user interface, local memory, and one or more communication components. The monitoring module 702 receives information from the monitoring device 703 and optionally makes that data available to the monitoring devices.

In one or more embodiments, the monitoring module may be in wired or wireless communication with a medical system or medical records database via communication with the same over a public or private data network. The medical system can optionally receive the biometric information detected by the monitoring device for analysis in conjunction to stored athlete information or medical records.

In one embodiment, the monitoring module 702 includes thresholds that can be set to generate alerts based upon certain types of data. For example, the monitoring module 702 can be configured to determine when a diagnostic, such as head acceleration for example, has exceeded a predetermined threshold. When this occurs, the monitoring module 702 can provide an alert that the acceleration event that exceeded the threshold. Such alerts can be useful in determining when a particular athlete has sustained a concussion. Similar alerts can be set for temperature, to alert when an athlete is overheating, for moisture, to alert when an athlete is becoming dehydrated, or based upon other monitored vital signs.

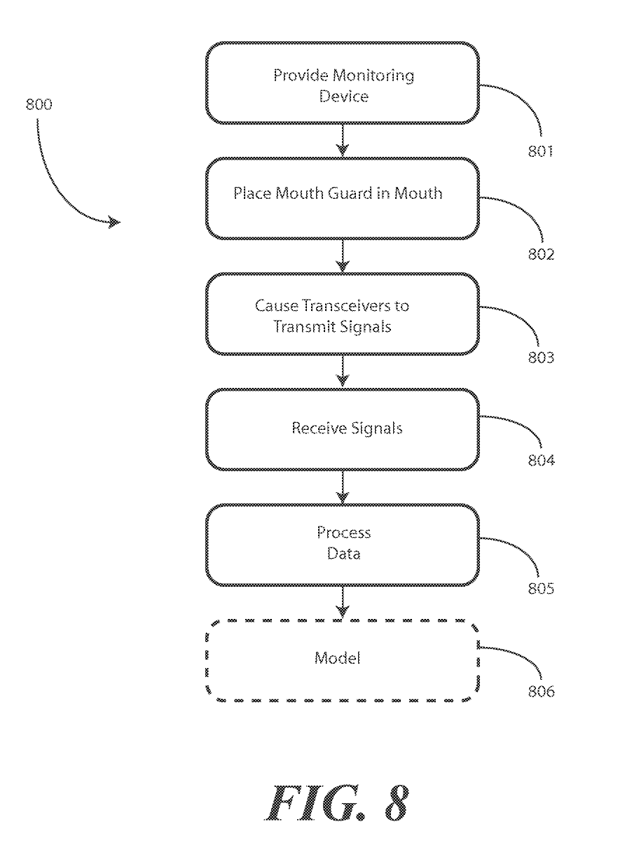

The general method steps used in the biometric monitoring system 700 of FIG. 7 are shown in FIG. 8. Turning now to FIG. 8, this method 800 is shown in flowchart form.

Beginning at step 801, a user to be monitored is provided a mouth guard comprising a biometric sensor and a switch operable to change a radar cross section of an antenna of the mouth guard by selectively altering a load impedance of the antenna. At step 802, the user places the mouth guard in their mouth and begins an activity.

Steps 803-806 then describe the steps of monitoring biometric activity of the user. At step 803, one or more transceivers transmit a radio frequency signal. At step 804, the one or more transceivers receives backscattered return signals having output of the biometric sensor encoded therein due to the switching of the switch in the mouth guard.

At step 805, the monitoring module receives data from the radio frequency transceiver. In one embodiment, the data comprises information corresponding to a linear force and/or a rotational force applied to the athlete. The monitoring module may normalize this data or otherwise process the same to determine impact information. Examples of impact information include a peak force, a rate of change of the force, and a magnitude or other characteristic of the force. The monitoring module may extrapolate acceleration and rotational forces from the data.

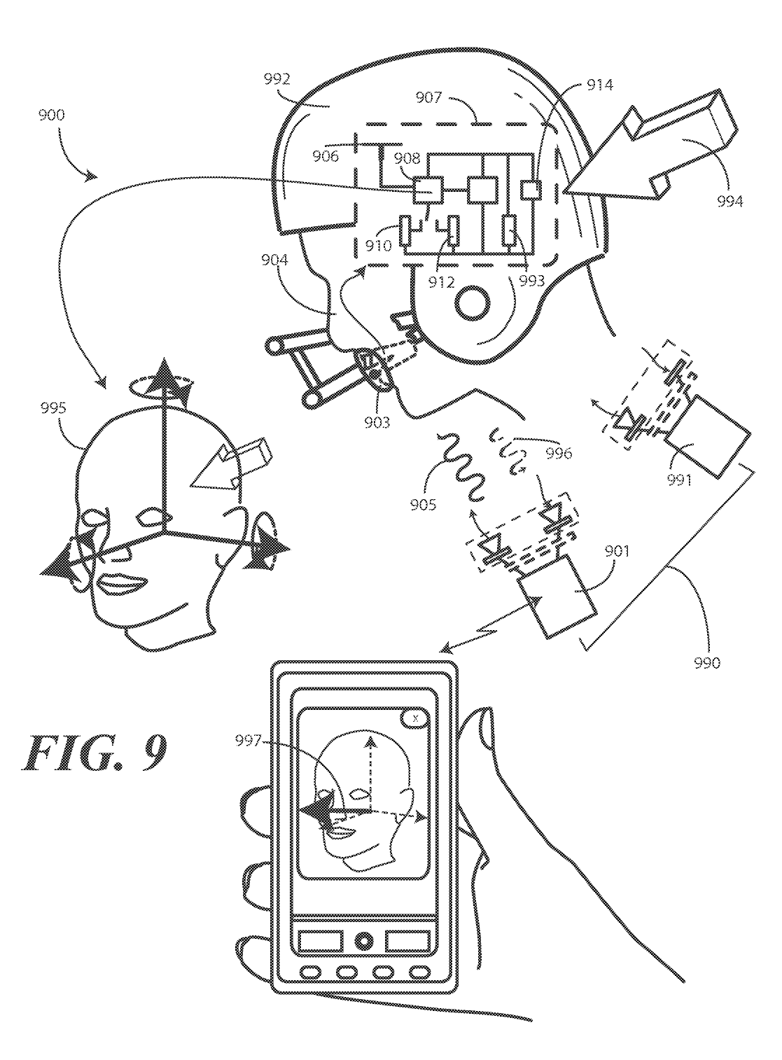

At optional step 806, a modeling module can use the processed data from the monitoring module to generate a model of the forces on a human skull. Such a model will be shown in more detail below in FIG. 9. For instance, the modeling module can translates the received biometric force data to a center of mass of a standardized human head, thus allowing for a model to be built that illustrates an effect of the impact on the head. In some cases the data will be algorithmically altered based upon the fact that the biometric sensors are disposed in the mouth of the athlete. Step 806 can also include presenting the data on one of the monitoring devices described above with reference to FIG. 7. For example, the generated model can be superimposed upon a graphical depiction of a human head. Alternatively, the modeling module can use the processed data to recreate the impact in the form of a video or a series of stills showing the event at different time intervals. Other modeling options will be obvious to those of ordinary skill in the art having the benefit of this disclosure.

While being described as generally applicable to monitoring athletes, those of ordinary skill in the art having the benefit of this disclosure will recognize that the method 800 of FIG. 8 can be used in other applications as well. For example, rather than configuring the device as a mouth guard, it may be configured as a bandage or sticker attached to the skin. Multiple monitoring devices can be attached to a subject at different locations. Accordingly, the method 800 of FIG. 8 can be used to monitor other data collection environments, such as data collection and modeling of the effects of a collision sustained during a car accident. Still other data collection environments will be obvious to those of ordinary skill in the art having the benefit of this disclosure.

Turning now to FIG. 9, illustrated therein is one embodiment of a system 900 similar to the biometric monitoring system (700) of FIG. 7 being used in practice. The system 900 of FIG. 9 includes a plurality of radio frequency transceivers 990, shown illustratively as radio frequency transceiver 901 and radio frequency transceiver 991. Each radio frequency transceiver 901,991 includes a monitoring module. A monitoring device 903, shown here as a mouth guard inserted into the mouth of an athlete 904. The athlete 904 of this illustrative embodiment is a football player.

The monitoring device 903 of this system 900 can be used to help diagnose concussions by measuring the linear and rotational accelerations on the football helmet 992 of the football player. Advantageously, embodiments of the present invention can accomplish this with greater accuracy than prior art systems, such as the HITS system manufactured by Simbex. In this illustrative embodiment, in addition to acceleration measurements, mouth temperature is measured with a temperature sensor, and heart rate is measured with exposed electrodes. Each of these sensors is configured as a biometric sensor of the monitoring device 903.

In this embodiment, the communication device 907 of the monitoring device 903 comprises a passive wireless device that communicates with the base station using wireless backscatter communications. In this embodiment, backscattering refers to the reflection of impinging signals with an alternating reflection coefficient signaling a message. A schematic block diagram of the components of the communication device 907, i.e., the components of the passive wireless device, is shown in FIG. 9.

Once powered, the baseband logic of a controller 908 of the communication device 907 reads the one or more biometric sensors of the monitoring device 903. A switch then modulates a reflector. When one or more of the radio frequency transceivers 901,991 needs to communicate information to the communication device 907 an optional envelope detector 993. The envelope detector 993 is configured to demodulate any commands sent to the communication device 907 by the radio frequency transceivers 901,991. In one embodiment, both the biometric sensor and the communication device 907 are integrated into the monitoring device 903 to prevent any corrosion from saliva or accidental swallowing.

The controller 908 and switch, which can be integrated in a single integrated circuit, causes the radar cross section of the antenna 906 to alternate between connections to two or more distinct reflective loads 910,912. The control and timing of the load modulator's switch is performed by the controller's baseband logic in accordance with signals received from the biometric sensor. In one embodiment, the controller's baseband logic can be implemented in a mixed-signal IC using complementary metal-oxide-semiconductor (CMOS) technology. A discrete logic device such as the Texas Instruments MSP430 series microprocessor can be used as well. The baseband logic device receives data from the biometric sensor, in one embodiment, in analog form using an analog-to-digital converter. In another embodiment, the baseband logic receives the data in digital form. For example, in one embodiment an STMicroelectronics LIS3LV02DQ linear accelerometer can be used as the biometric sensor to communicate acceleration measurements digitally via a data connection to the controller 908.

In this illustrative embodiment, to provide a completely passive communication device, an energy harvester 914 is included as the power source. The energy harvester 914 converts ambient energy from the environment into electrical energy for the components of the monitoring device 903. Examples of suitable energy harvester devices include a radio frequency rectifier or charge pump, a vibration harvester, or acoustic harvester. The envelope detector 993, where included, is typically a rectifying circuit that is built from diodes and capacitors. The baseband logic of the controller 908, the energy harvester 914, the envelope detector 993, and even the biometric sensor, be it one or more accelerometers, one or more gyroscopes, or one or more other sensors can all be contained within an integrated circuit. However, a discrete component implementation is feasible as well.

As shown in FIG. 9, the antenna 906 of the communication device 907 receives incoming signals 905 from a radio frequency transceiver 901 and backscatters reflected signals 996. A typical antenna 906 can be configured to have a gain pattern that aims outside the mouth (as opposed to aiming to the throat) with circular or elliptical polarization. This type of gain pattern is one flexible and reliable configuration that ensures signals are backscattered when the monitoring device 903 is turned upside down or sideways as a result of the physical activity or a severe hit. Other antenna types with multiple elements, linear polarization, or different gain patterns are possible as well for use as antenna 906. The planar-F antenna is another suitable example since they are designed to work near human flesh. A near-field antenna is feasible as well since it is nearly impervious to attenuation of the flesh of the lips and cheeks. Many configurations and topologies of antennas are feasible, but in one embodiment a far-field, circularly polarized antennas with a wide gain pattern aiming outside the mouth is used. Note that some mouth guard shapes have material that protrudes out from the mouth such as that shown above in FIGS. 5 and 6. Far-field antennas implanted in the mouth guard outside the mouth can be more efficient since the backscattered signals do not have to pass through the lips.

The plurality of radio frequency transceivers 990 provide an unmodulated incoming signal 905. Energy from the incoming signals 905 is then harvested for DC power by the energy harvester 914. The antenna 906 then modulates and reflects a backscattered return signal 996. There is a wide variety of signal configurations that can be used, but in one embodiment, the incoming signals 905 and the backscattered return signals 996 correspond to the rules of the Federal Communications Commission (FCC) for the designed frequency band. Any of the radio frequency transceivers 901,991 can transmit incoming signals 905 multiple times per second. The monitoring module 902 of one of the radio frequency transceivers 901 can analyze received data, and then save and report the data to the user or a medical professional, coach, or personal trainer.

The frequency bands used for communications will typically be an industrial, scientific, and medical (ISM) band, which allow use of the spectrum by unlicensed users by the FCC. Possible frequency bands and their communications standards may include, but are not limited to:

ISO 18000-3: Air interface standard for 13.56 MHz;

ISO 18000-4: Air interface standard for 2.45 GHz;

ISO 18000-6: Air interface standard for 860 to 940 MHz;

ISO 18000-7: Air interface standard for 433.92 MHz;

IEEE 802.15.1: “Bluetooth” standard for 2.4 GHz;

IEEE 802.15.1: Wireless personal area networks coexisting with wireless local area networks at 2.4 GHz or 5.8 GHz;

IEEE 802.15.4: Low-rate wireless personal area networks for semi-passive tags or long-battery life tags on which the Zigbee specification is based; and

Dash-7: tags consuming less than 1 mW operating at 433.92 MHz.

As shown in FIG. 9, a large arrow 994 is representative of a hit incurred during activity.

In this embodiment, forces from the hit are measured in a three-dimensional Cartesian coordinate system used to measure both linear and rotational acceleration. As indicated by the direction of the arrow 994, the biometric sensor will detect significant linear acceleration components in the negative y and z directions. In addition, the head of the athlete 904 will experience significant rotational acceleration in the negative z direction and slight rotation in the positive y direction from this force. The monitoring module 902 receives this data and models the impact on a coordinate system and head 995. This information, which includes a resulting vector 997, can then be presented on a monitoring device.

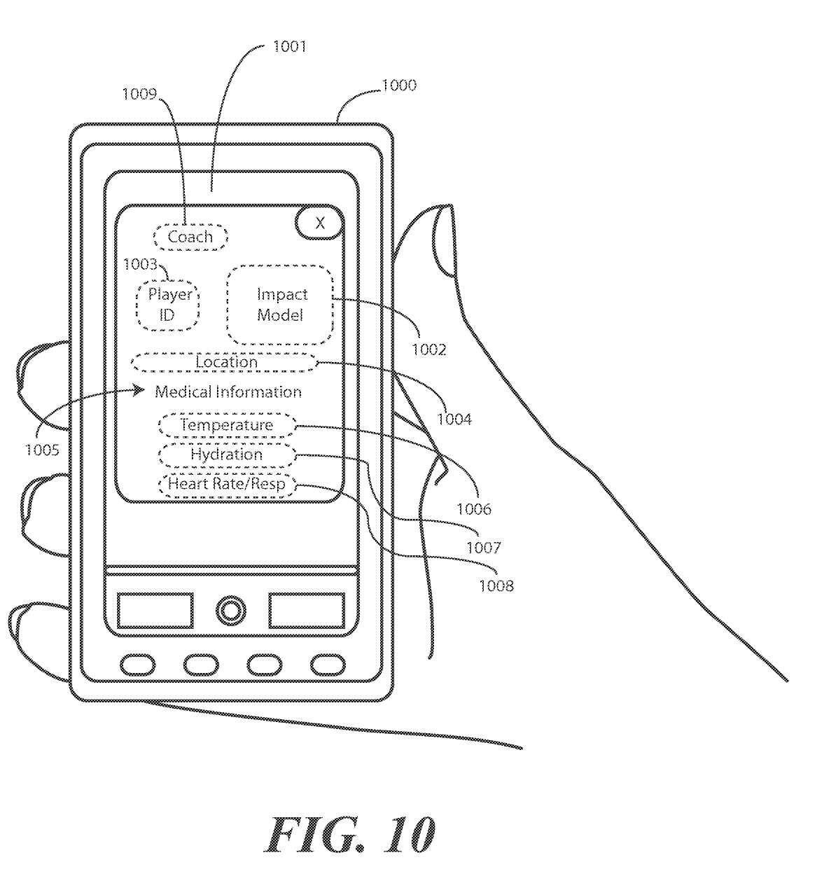

Turning now to FIG. 10, an explanatory output of systems described herein is illustrated on the display 1001 of a mobile device 1000. As noted in the discussion of FIG. 9, modeled impact information 1002 can be presented on the display 1001. The identity 1003 of the athlete can be presented in accordance with identification methods that will be described in the figures that follow, as can location information 1004 indicating where the athlete is on the playing field.

In one or more embodiments, medical information 1005 received from a medical service as described above that corresponds to the athlete can be displayed as well. Other biometric information, including temperature information 1006, hydration information 1007, and heart rate and/or respiration information 1008 can also be presented.

In one or more embodiments, information 1009 identifying the coach, trainer, or parent can be presented as well. The modeled impact information 1002 can be shown in analog or numeric form, depending upon which is more efficient at informing a coach, trainer, or health care provider the magnitude of the most recent impact. In one or more embodiments, the same information can be delivered to a server or other device disposed near the playing field.

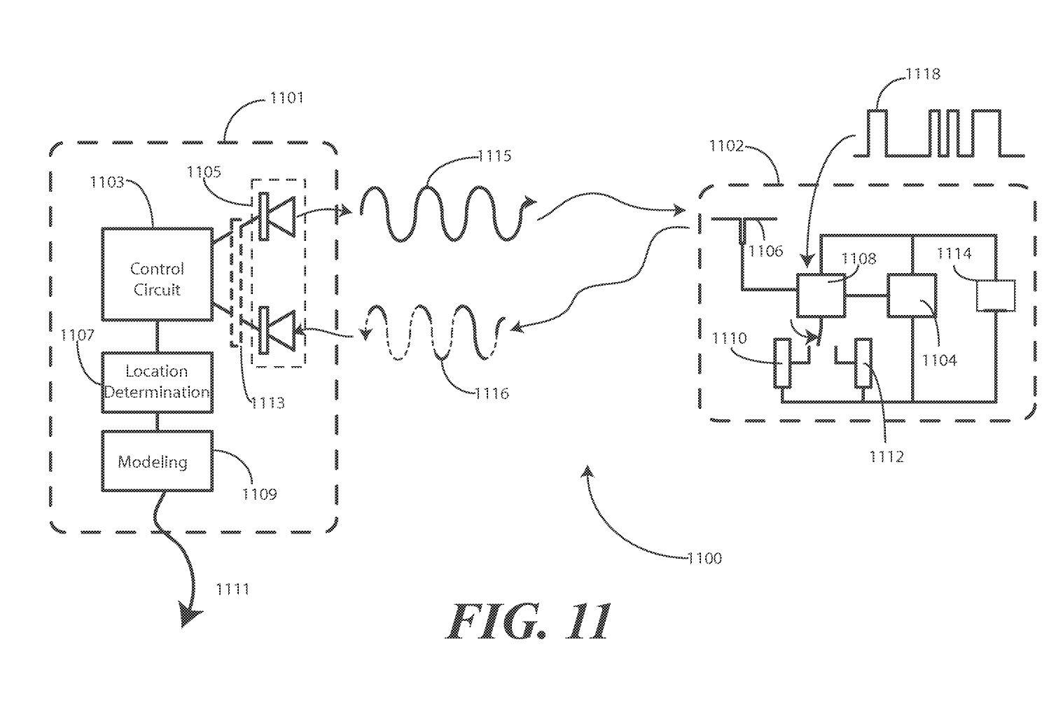

To this point, biometric monitoring has been described. However, as noted in the discussion of the information that can be presented on the display 1001, in one or more embodiments, identification and location of a particular player can be presented as well. Location and identification information can be especially useful when multiple monitoring devices are being deployed. For example, in a typical football game, there may be 100 or more monitoring devices being used, with 22 on the field at any one time. Accordingly, it can be advantageous to be able to identify individual players when monitoring biometric data. FIGS. 11-16 illustrate how location and identification can be determined. The devices of FIGS. 11-16 can be included with the biometric sensors described above to provide data corresponding to biometrics, identity, location, and anatomical modeling. Such modeling is described in copending application Ser. No. 12/505,916.

Turning first to FIG. 11, illustrated therein is a radio transceiver 1101 and alternate communication device 1102. The illustrative radio transceiver 1101 is similar to that described above with reference to FIG. 4 and includes a control circuit 1103 and a transceiver 1105. The communication device 1102 includes a controller 1104, an antenna 1106, and a switch 1108 that is responsive to the controller 1104. The switch 1108 is operative to change a radar cross section of the antenna 1106 by selectively altering a load impedance of the antenna 1106. As with the embodiment of FIG. 4, in the illustrative embodiment of FIG. 11, the switch 1108 does this by selectively switching between two loads 1110,1112 coupled to the antenna 1106.

The transceiver 1105 of the radio transceiver 1101 is responsive to the control circuit 1103 and is configured to transmit a radio frequency signal 1115. Where multiple radio transceivers are used in an application, the radio frequency signal 1115 transmitted by each radio transceiver may be unique. For example, in one embodiment, each radio frequency signal 1115 transmitted may be offset from the others by a predetermined phase. In another embodiment, each radio frequency signal 1115 transmitted may have a different frequency or characteristic waveform.

The communication device 1102 receives this radio frequency signal 1115 at its antenna 1106. In one embodiment, the antenna 1106 comprises a slot antenna suitable for receiving radio frequency communication. The switch 1108 of the communication device 1102 then, in response to a control signal from the controller 1104, switches. In the embodiment above shown in FIG. 4, this switching was in response to a biometric sensor. In FIG. 11, the switching is in accordance with both information received from a biometric sensor and in accordance with a unique identification code 1118 stored in the controller 1104. This switching serves to modulate or encode both the biometric information and the unique identification code 1118 into a reflected return signal 1116 that is backscattered from the antenna 1106. The transceiver 1105 then receives this backscattered return signal 1116 having the biometric data and the unique identifier modulated therein. By reading the unique identifier, the control circuit 1103 is able to determine from which communication device 1102 the backscattered return signal 1116 was reflected or backscattered.

A location determination module 1107, which may be configured in software as executable code or in hardware as programmable logic, is then configured to compare the received backscattered return signal 1116 with the transmitted radio frequency signal 1115 to make location determination estimates. In one embodiment, the location determination module 1107 is configured to determine the location of the communication device 1102 by determining a phase shift between the transmitted radio frequency signal 1115 and the backscattered return signal 1116 to determine a distance between the communication device 1102 and the radio transceiver 1101. In another embodiment, the location determination module 1107 is configured to determine a signal strength of the backscattered return signal 1116 and compare it with the signal strength of the transmitted radio frequency signal 1115 to determine a distance between the communication device 1102 and the radio transceiver 1101. Where multiple radio transceivers are disposed about the area of interest, these distances can be used in a triangulation method to determine a location estimation of each communication device.

In one embodiment, the location determination module 1107 is configured to determine both a first location determination and a second location determination. The first location determination can be a coarse location estimate, while the second location determination can be a fine location estimate. Each location determination can be made using the same backscattered return signals 1116. For example, presuming three or more radio transceivers are disposed about an area of interest, in one embodiment the first location determination can be made by triangulating distances from the three or more radio transceivers using the signal strength of each backscattered return signal received by each radio transceiver. In one embodiment, the second location determination can be made by triangulating distances from the three or more radio transceivers using the phase shift between transmitted radio frequency signals and the backscattered return signals received by each radio transceiver.

Where multiple radio transceivers are used, the corresponding control circuits can be combined into a single control circuit or may otherwise be integrated into a single device. For example, each radio transceiver 1101 may include an output 1111 suitable for coupling to a general-purpose computer, application specific device, or user interface.

Where multiple radio transceivers 1101 are used to determine the location of any one communication device 107, in one embodiment each radio transceiver 1101 is capable of receiving a backscattered return signal 1116 from each other radio transceiver. Said differently, while radio transceiver 1101 may emit its own, unique radio frequency signal 1115, it may receive backscattered return signals from multiple other radio transceivers. This configuration can have advantages in some applications, as advanced location determination techniques can be applied to the plurality of received signals.

In other applications, however, it may be desirable to only receive a return signal that corresponds to the signal delivered from the transceiver 1105. One way to accomplish this is by including an optional filter 1113 configured to pass some backscattered return signals while blocking others. For example, where each radio transceiver transmits a radio frequency signal of a different frequency, the radio transceiver 1101 can be equipped with the optional filter such that only the backscattered return signal 1116 having the unique identifier modulated therein that corresponds to the radio frequency signal 1115 transmitted by radio transceiver 1101 will be received, as other backscattered return signals will be blocked.

In another embodiment, such as to reduce multipath distortion, the optional filter 1113 can be configured to block signals that are unmodulated, while passing those that have been modulated by the communication device 1102. Such a filter 1113 helps to reduce both noise and distortion that can affect location determination.

Where multiple radio transceivers are used, and further where multiple communication devices are used, one or more of the radio transceivers may include an optional object modeling module 1109. The object modeling module 1109 may be configured in software as executable code or in hardware as programmable logic. While shown in FIG. 11 as being incorporated into one of the radio transceivers 1101, the object modeling module 1109 may be separate from each of the radio transceivers. Additionally, the object modeling module 1109 may be a component disposed in a general-purpose computer or application specific device that is coupled to one or more of the radio transceivers via the output 1111.

In one embodiment, the object modeling module 1109 is configured to model a multi-dimensional shape of an object. Recall from above that while the communication device 107 can be integrated into a mouth guard, it can also be integrated into other devices, including bandages, or stickers for application to the skin. It can also be integrated into pads and equipment, shoes or other objects. Presuming that a subject has a plurality of monitoring devices is disposed along their body, the object modeling module 1109 can map the shape of the object from the determined location of each monitoring device, using an interpolation algorithm to create surfaces between the location of each monitoring device. For example, in one embodiment, the object modeling module 1109 can map the shape of the object by linearly connecting the locations of each monitoring device. In another embodiment, a higher order function may be used to connect the monitoring device locations to form the multidimensional shape of the object.

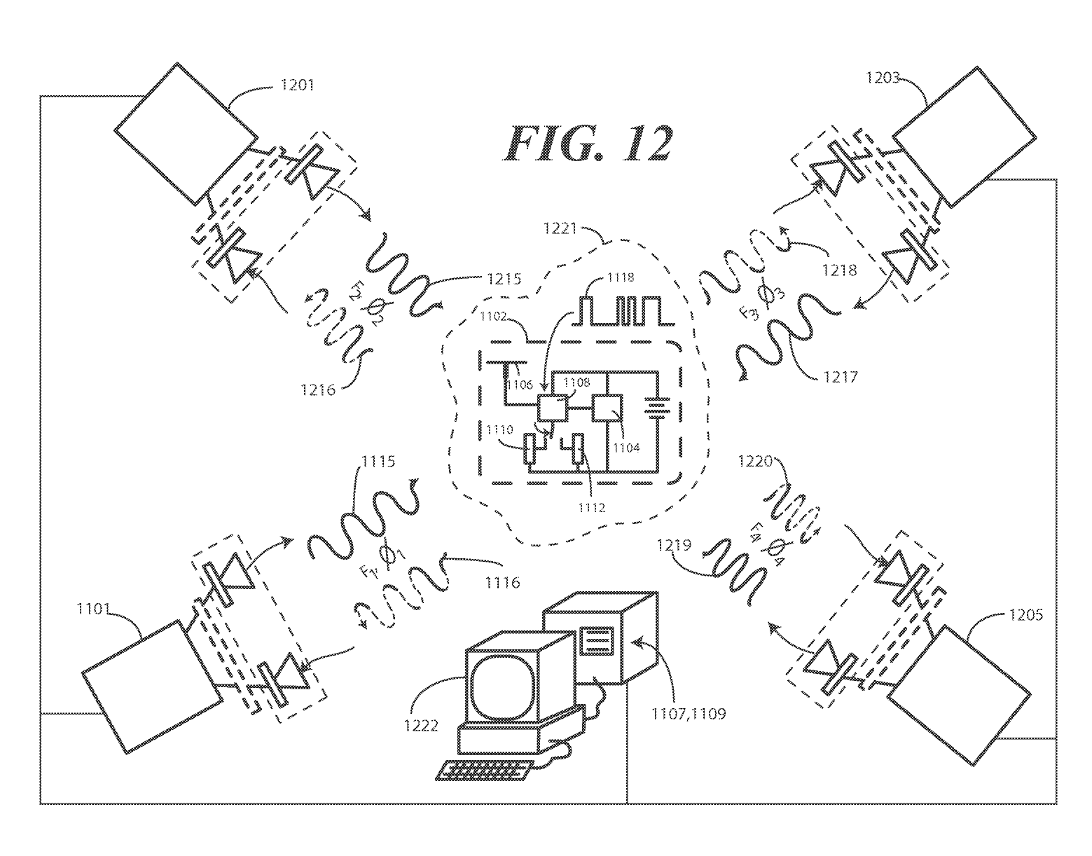

Turning now to FIG. 12, illustrated therein is one embodiment of a reflected interferometry system 1200 employing multiple radio transceivers 1101, 1201, 1203, 1205 to determine the location of one or more monitoring devices having the previously described communication device 1102 in free space. In the illustrative embodiment of FIG. 2, there are four radio transceivers 1101, 1201, 1203, 1205. While three radio transceivers can be used, many applications will benefit from at least four radio transceivers where relatively accurate course location estimates are desired. Further, the use of more radio transceivers tends to make the overall system 1200 more resistant to multiple signal paths and blockage. This is true because the power and phase measurements described above are made from modulated reflections from the communication device 1102. As such, much of the multipath distortion received by a radio transceiver can be filtered out - since it is unmodulated - by the optional filter (1113). Further, adding additional radio transceivers provide redundancy such that the location determinations can be made even when one radio transceiver fails to receive a signal.

In the illustrative embodiment of FIG. 12, the radio transceivers 1101, 1201, 1203, 1205 are disposed about a location of interest 1221. In one embodiment, each radio transceiver 1101, 1201, 1203, 1205 includes a transmitter that is separate and distinct from a receiver. The transmitter of each radio transceiver 1101, 1201, 1203, 1205 can be configured to transmit a radio frequency signal 1115, 1215, 1217, 1219. In one embodiment, the radio frequency signals 1115, 1215, 1217, 1219 are transmitted continuously while location determination is occurring.

As also noted above, in one embodiment each radio frequency signal 1115, 1215, 1217, 1219 is unique. For example, each radio frequency signal 1115, 1215, 1217, 1219 may have its phase or frequency offset from each of the other radio frequency signals as indicated in FIG. 12.

The communication device 1102 of the present invention is unique, in that it backscatters each of the radio frequency signals 1115, 1215, 1217, 1219 by switching between two loads 1110,1112 coupled to the antenna 1106. This communication device 1102 is inexpensive to manufacture in that it does not require any RF components such as matching circuits, transmission lines, and the like. Its largest component is generally the antenna 1106. However, the antenna 1106 merely receives and reflects incident power from each radio frequency signal 1115, 1215, 1217, 1219, thereby modulating a unique identifier associated with the communication device 1102 (as well as the biometric data from a biometric sensor (not shown)) into the backscattered return signals 1116, 1216, 1218, 1220. The physical form factor of the antenna 1106 is scalable with frequency. Experimental testing has shown that a 5.7 GHz antenna can be manufactured to be 2 centimeters or less in length.

In the illustrative embodiment of FIG. 12, the controller 1104 of the communication device 1102 is configured to continually cause the switch 1108 to switch between at least two loads 1110,1112 in accordance with a unique identification code 1118 and biometric data. This continuous switching accordingly changes the radar cross section of the antenna 1106 of the communication device 1102 in accordance with the unique identification code 1118 and biometric information. As such, when each radio frequency signal 1115, 1215, 1217, 1219 is received by the antenna 1106, it is backscattered as a plurality of backscattered return signals 1116, 1216, 1218, 1220 to the radio transceivers 1101, 1201, 1203, 1205. Each backscattered return signal 1116, 1216, 1218, 1220 has the unique identifier and biometric data modulated therein, thereby allowing each radio transceiver 1101, 1201, 1203, 1205 to identify from which monitoring device it was sent, even where there are numerous monitoring communication devices in the location of interest 1221 or field of view. Research suggests that as many as 256 different sensors or more can be disposed within the location of interest 1221 without degrading system performance This number is more than ample for most all competitive sports.

In the illustrative embodiment of FIG. 12, each radio transceiver 1101, 1201, 1203, 1205 is coupled to a computer 1222 having some components the location determination module 1107 operational therein. Other components of the location determination module 1107 are operational in each of the radio transceivers 1101, 1201, 1203, 1205. The location determination module 1107 knows the locations of the radio transceivers 1101, 1201, 1203, 1205 accurately. In the illustrative embodiment of FIG. 12, the computer 1222 also has the optional object modeling module 1109 operating therein.

Upon reflection, each backscattered return signal 1116, 1216, 1218, 1220 travels to the receiver of each radio transceiver 1101, 1201, 1203, 1205. When each radio transceiver 1101, 1201, 1203, 1205 receives each backscattered return signal 1116, 1216, 1218, 1220, components of the location determination module 1107 (neglecting operation the biometric processing components since they have been described above) operating in each radio transceiver 1101, 1201, 1203, 1205 may determine a distance between the radio transceiver and the communication device 1102. As noted above, this can be done in various ways.

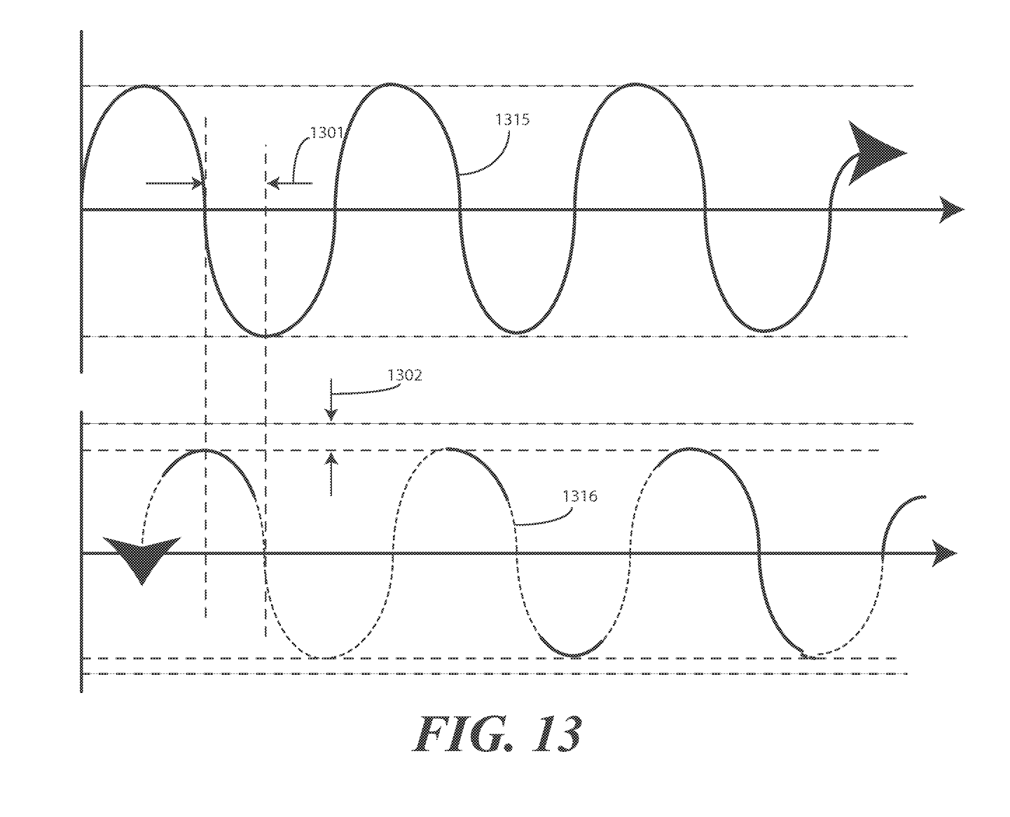

Turning now briefly to FIG. 13, in one embodiment, the location determination module (1107) can determine the distance by determining a phase shift 1301 between the transmitted radio frequency signal 1315 and the corresponding received backscattered return signal 1316. In another embodiment, the location determination module (1107) can determine the distance by determining a signal strength 1302 of the backscattered return signal 1316. Of course, as noted above, combinations of the two approaches can be used.

Turning now back to FIG. 12, in this illustrative embodiment the distance measurement determinations can then be delivered to the components of the location determination module 1107 operating in the computer 1222. Those components can then determine the location of the communication device 1102 by triangulating the distances received from each radio transceiver 1101, 1201, 1203, 1205. As noted above, the triangulation can be performed by using the signal strength of each backscattered return signal 1116, 1216, 1218, 1220, or by using the phase shift between the transmitted radio frequency signals 1115, 1215, 1217, 1219 and the backscattered return signals 1116, 1216, 1218, 1220. In one embodiment, the components of the location determination module 1107 operating in the computer are capable of determining a first location determination, which is a course estimate, using signal strength. The components are also capable of determining a second location determination, which is a fine estimate, using phase.

In one embodiment, the first location determination is based upon received signal strength fingerprinting technology. Radio transceivers 1101, 1201, 1203, 1205 that are closer to the communication device 1102 receive stronger signals, while more distant radio transceivers 1101, 1201, 1203, 1205 receive weaker signals. Each communication device 1102 has a unique combination of signal strengths that can be used to provide the location estimate. Further, the backscatter link is lossier than conventional free-space wireless links. The additional propagation loss can be used it increase accuracy determination when the signals are triangulated. Such technology is known in the art and has been used, for example, in cellular communication systems to determine the location of a caller dialing 911. Such technology is illustratively set forth in a paper by N. Patwari, A. Hero III, M. Perkins, N. Correal, and R. O'Dea, entitled “Relative Location Estimation in Wireless Sensor Networks,” IEEE Transactions on Signal Processing, vol. 51, no. 8, pp. 2137-48, August 2003, http://dx.doi.org/10.1109/TSP.2003.814469, which is incorporated herein by reference. Location determination based upon signal strength measurements is also illustratively described in a paper by R. Yamamoto, H. Matsutani, H. Matsuki, T. Oono, and H. Ohtsuka, entitled “Position Location Technologies Using Signal Strength in Cellular Systems,” IEEE VTS 53rd Vehicular Technology Conference, Spring, 2001. Proceedings (Cat. No. 01CH37202), vol. vol. 4, pp. 2570-4, 2001, http://dx.doi.org/10.1109/VETECS.2001.944065, and a paper by J. Zhu, Indoor/Outdoor Location of Cellular Handsets Based on Received Signal Strength,” Georgia Tech PhD Dissertation, June 2006, http://www.propagation.gatech.edu/Archive/PG_TR_{—}060515_JZ/PG_TR_{—}060515_JZ.pdf, both of which are incorporated herein by reference. The system 1200 of FIG. 12 offers advantages over the prior art in that multi-path return signals and free-space blockages do not degrade system performance

A second, more accurate location estimate can be achieved using phase shift between the transmitted radio frequency signals 1115, 1215, 1217, 1219 and the backscattered return signals 1116, 1216, 1218, 1220. Each radio transceiver 1101, 1201, 1203, 1205 has a corresponding signal path defined between it and the communication device 1102. The path from each radio transceiver 1101, 1201, 1203, 1205 to the communication device 1102 will introduce a phase change in the transmitted and backscattered wave that is proportional to the total path link. For example, if the signal path from radio transceiver to communication device is 5.83 meters, and the radio frequency being used is 5.7 GHz, 3 centimeters of phase difference will be introduced into as the wave travels from the radio transceiver to the communication device. This corresponds to a phase difference of 144 degrees. A corresponding amount of phase difference will be introduced on the return trip. Thus, each radio transceiver 1101, 1201, 1203, 1205 will measure a different amount of phase shift due to the location of the communication device 1102.

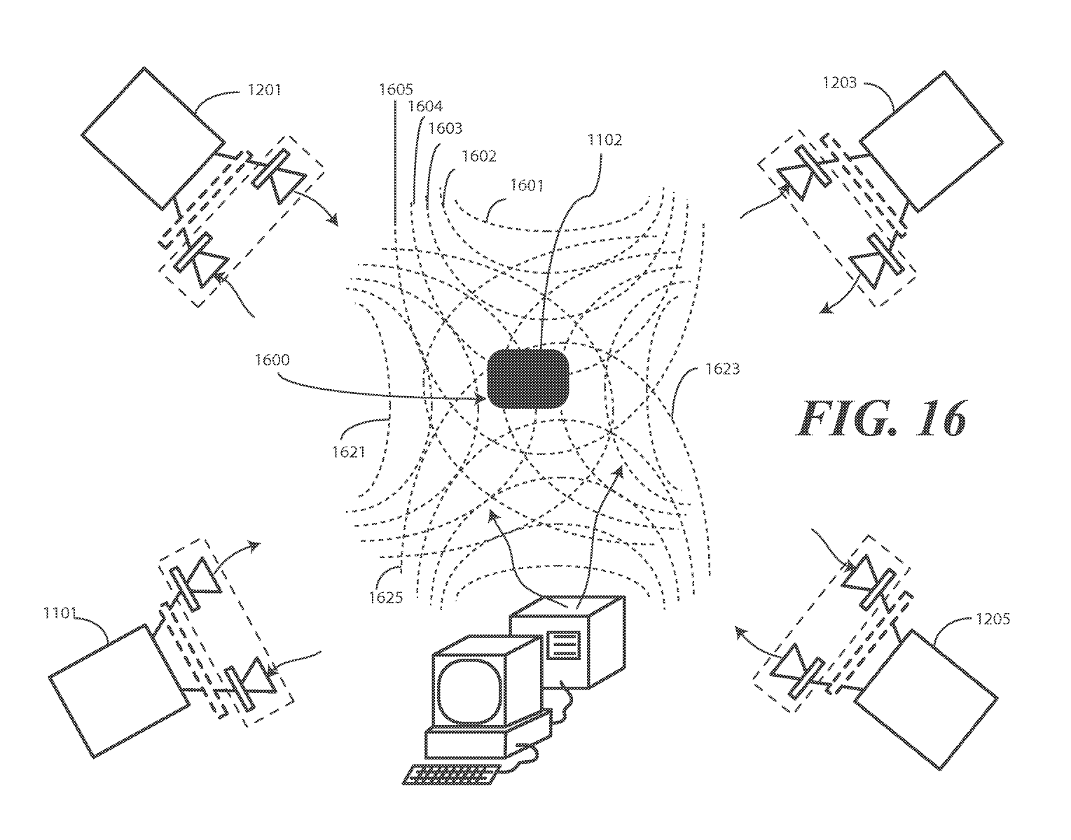

Turning briefly to FIG. 16, in one embodiment the location determination module can use the determine phase shift to determine the location of the communication device 1102 by modeling a series of hyperboloids 1601,1602,1603,1604,1605 that correspond to a signal path associated with the measured phase difference. As each radio transceiver 1101, 1201, 1203, 1205 has a series of hyperboloids, e.g., hyperboloids 1601,1601,1603,1605, corresponding thereto, the intersection 1600 of each indicates the location of the communication device 1102.

To illustrate by way of example, a phase difference measurement at 5.7 GHz at 72 degrees would yield a hyperbolic surface in three dimensions that is indicative of a total path length change of 4.36 meters from radio transceiver to communication device. Neighboring hyperbolic surfaces would correspond to total path lengths of 4.31 meters and 4.41 meters, respectively.

Location of the communication device 1102 can then be resolved by using multiple phase measurements from the multiple backscattered return signal measurements. Each phase measurement results in a series of hyperbolic surfaces that can be intersected with others to eventually produce a reliable and sufficiently accurate location estimation of the communication device 1102.

Using the four-radio transceiver system shown in FIGS. 12 and 16, the phase change between radio transceiver 1101 and radio transceiver 1205 may be modeled as a set of hyperboloids 1625. When intersected with hyperboloids 1623 modeling the phase change between radio transceiver 1203 and radio transceiver 1205, the location of the communication device 1102 can be narrowed to a set of lines in three-dimensional space, or a set of points in two-dimensional space. When further intersections are made with the hyperboloids 1621 modeling the phase change between radio transceiver 1101 and radio transceiver 1201, the location of the communication device 1102 is narrowed to a series of points in three-dimensional space, or to a specific point in two-dimensional space. In three-dimensional space, a fourth set of hyperboloids modeling the phase change between radio transceiver 1201 and radio transceiver 1203 pinpoints the location of the communication device 1102. Additional radio transceivers may be used for redundancy or reliability.

Turning back to FIG. 12, in one embodiment, the location determination module 1107 is configured to negate any phase changes introduced by the antenna 1106 of the communication device 1102. This is done to increase the overall accuracy of the system 1200. However, in many applications, the phase change introduced by the antenna 1106 will not be large enough to adversely affect the location determination.

In one embodiment, the location determination module 1107 is configured to determine location both from the course location estimate using signal strength and the fine location estimate using phase change. By using both determination methods, the effects of noise, interference, and multiple signal paths can be overcome. For example, using the course location estimate, a “sphere of likelihood” can first be determined. Next, the hyperboloids of the fine location estimate may only be drawn in within the sphere of likelihood, thereby reducing the computation associated with an accurate location determination.

In one embodiment, where both fine and course location determinations are used, a carrier frequency for the radio frequency signals 1115, 1215, 1217, 1219 will be selected such that the sphere of likelihood determined from the course location determination would include 3 or 4 hyperboloids. Generally speaking, lowering the carrier frequency of the radio frequency signals 1115, 1215, 1217, 1219 results in longer wavelengths, which in turn leads to hyperboloids that are father apart.

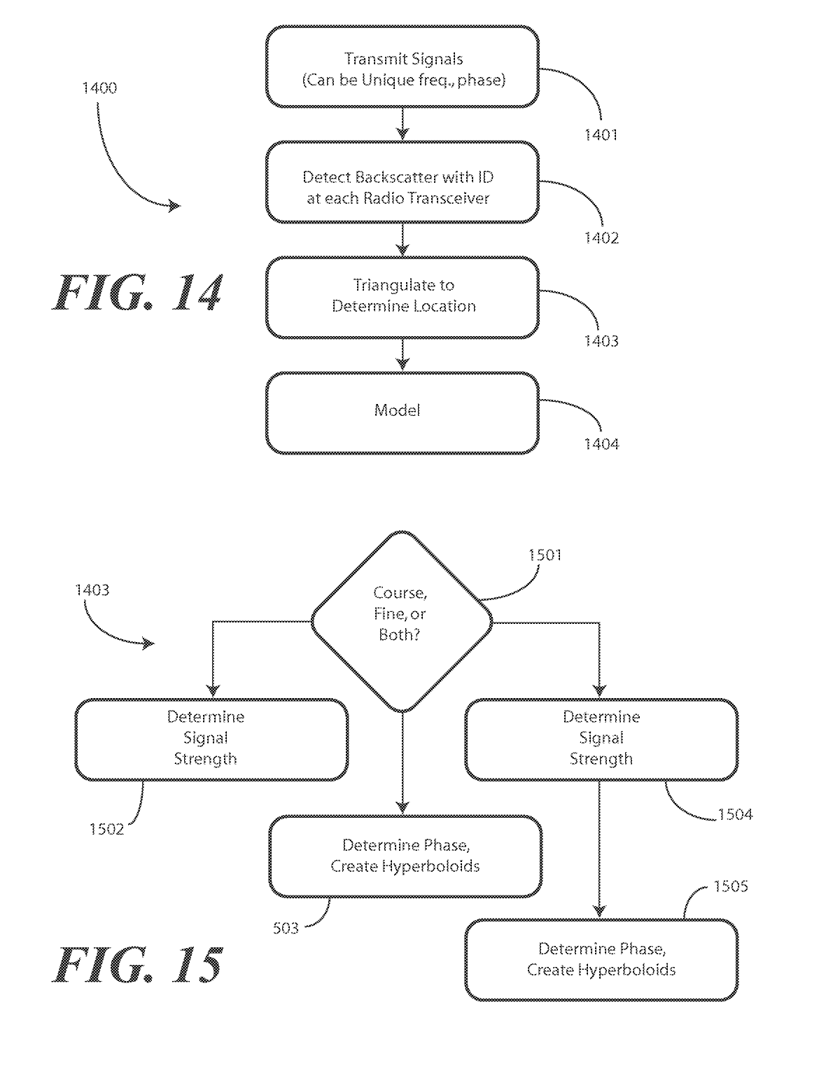

Turning now to FIG. 14, illustrated therein is one embodiment of a method 1400 for determining the location of an object using reflected interferometry in accordance with embodiments of the invention. The method 1400 of FIG. 4 can be configured as executable instructions to be stored in a computer readable medium, such as a memory device, for controlling the control circuit and location determination module to execute some or all of the functions of determining the location of an object in free-space as described herein. Alternatively, the method could be carried out by application specific hardware devices or programmable logic as well. Moreover, the method 400 can be used in conjunction with the method (800) of FIG. 8 to provide combined location and biometric information to a monitoring device.

At step 1401, a plurality of radio frequency signals are transmitted from a plurality of radio transceivers. In one embodiment, the radio frequency signals are transmitted continuously while the method 1400 is being executed, although intermittent transmission is also possible. In one embodiment, the radio frequency signals are transmitted on a one-to-one basis from each radio transceiver, such that each radio transceiver transmits one radio frequency signal. In one embodiment, four radio transceivers are used to transmit four radio frequency signals into an area of interest. The radio frequency signals can each be unique. For example, in one embodiment, each radio frequency signal is transmitted with one of a unique frequency, a unique phase shift, or combinations thereof.

At step 1402, each radio transceiver receives one or more backscattered return signals from one or more communication devices disposed within one or more biometric monitoring devices. As described above, in one embodiment, each communication device has a switch capable of selectively reflecting and modulating each of the transmitted radio frequency signals to encode an identifier that is unique to the communication device, and also to encode biometric data, therein. In one embodiment, the switch toggles between a high impedance load and a low impedance load in accordance with a unique identification code and/or biometric data to modulate the identifier and biometric information into the radio frequency signal and reflect and return it to the plurality of transceivers as one or more backscattered return signals.

At step 1403, the method 1400 receives the biometric data and further determines the location of the one or more communication devices from information derived from the one or more backscattered return signals. For example, in one embodiment, the method 1400 uses triangulation techniques to determine the location of the one or more communication devices at step 1403. As shown in FIG. 15, the location determination technique of step 1403 can be accomplished in a variety of ways. Specifically, as determined at decision 1501, the location determination step can include a course location determination at step 1502, a fine location determination at step 1503, or a combination of the two as shown in steps 1504 and 1505.

At step 1502 and step 1504, as described above, the triangulation can be performed using the relative signal strengths of the one or more backscattered signals to achieve a coarse location estimate. The signal strength of the backscattered signal can be compared to the signal strength of the transmitted radio frequency signal. As each radio transceiver determines a different relative signal strength, these differences can be triangulated to obtain a course location estimate.

At step 1503 and step 1505, as also described above, triangulation could be performed by generating a series of hyperboloids modeling the distances between each radio transceiver and each communication device to achieve a fine location estimate, where those distances are determined from a phase shift detected between each transmitted radio frequency signal and the corresponding backscattered return signal. The phase difference of the backscattered return signal, when compared to the transmitted radio frequency signal, can be used to generate the hyperboloids. The combination of the two approaches can also be used as illustrated at steps 1504,1505. One example of this is the method using the course location estimate to determine a sphere of probability with the fine location estimate pinpointing the actual location of a communication device within the sphere noted above.

Where multiple communication devices are employed, it can sometimes be advantageous to model the shape of the object to which the communication devices are affixed. For example, embodiments of the invention are well suited for biomechanical sensing, such as athletic activity, as many monitoring devices can be coupled to an athlete executing a biometric motion. As the radio transceivers of embodiments of the present invention are capable of determining the locations of each communication device while the biometric motion is being executed, it can be useful to form a visual model of the athlete by modeling surfaces between the communication devices to approximate the student on a video screen. This is especially useful for review of collisions and other impact events occurring in contact sports. Turning now back to FIG. 14, at step 1404, the method uses the knowledge of the locations of all the communication devices to model the shape of the object to which the communication devices are affixed. The modeling, in one simple embodiment, may just be a linear interpolation between each communication device, which is represented on a video screen by a straight line. In a more complicated embodiment, three-dimensional surfaces can be modeled between the communication device locations to create a more accurate representation of the object.

In the foregoing specification, specific embodiments of the present invention have been described. However, one of ordinary skill in the art appreciates that various modifications and changes can be made without departing from the scope of the present invention as set forth in the claims below. Thus, while preferred embodiments of the invention have been illustrated and described, it is clear that the invention is not so limited. Numerous modifications, changes, variations, substitutions, and equivalents will occur to those skilled in the art without departing from the spirit and scope of the present invention as defined by the following claims. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

What is claimed is:

1. An electronic monitoring device, comprising:
a mouth guard;
a biometric sensor attached to or integrated with the mouth guard; and
a communication module, operable with the biometric sensor and comprising:
an antenna;
a controller; and
a switch, responsive to the controller, and operative to change a radar cross section of the antenna by selectively altering a load impedance of the antenna;
wherein the controller is configured to backscatter received radio frequency signals by modulating the received radio frequency signals by controlling the switch to encode output from the biometric sensor into a backscattered return signal.

2. The electronic monitoring device of claim 1, wherein the backscattered return signal comprises a multi-path backscattered return signal.

3. The electronic monitoring device of claim 1, wherein the biometric sensor comprises a linear acceleration detector.

4. The electronic monitoring device of claim 1, wherein the biometric sensor comprises a rotational acceleration sensor.

5. The electronic monitoring device of claim 1, wherein the biometric sensor comprises a temperature sensor.

6. The electronic monitoring device of claim 1, wherein the biometric sensor comprises a moisture sensor.

7. The electronic monitoring device of claim 1, wherein the biometric sensor comprises a heart rate sensor.

8. The electronic monitoring device of claim 1, wherein the biometric sensor comprises a respiration sensor.

9. The electronic monitoring device of claim 1, further comprising biometric sensor terminals having one or more surfaces exposed to a surface of the mouth guard.

10. The electronic monitoring device of claim 1, wherein the mouth guard defines a channel configured to receive a user's lips, wherein the biometric sensor is disposed on a first side of the channel and the communication module is disposed on a second side of the channel.

11. The electronic monitoring device of claim 1, wherein the controller is further configured to modulate the received radio frequency signals by controlling the switch to additionally encode a unique identifier into the backscattered return signal.

12. The electronic monitoring device of claim 1, wherein the communication module comprises an energy harvester configured to use energy from the received radio frequency signals to power the communication module.

13. The electronic monitoring device of claim 12, wherein the energy harvester is further configured to power the biometric sensor.

14. The electronic monitoring device of claim 1, further comprising a generator configured to power one or more of the biometric sensor, the communication module, or combinations thereof.

15. A biometric monitoring system, comprising:
one or more radio frequency transceivers;
a monitoring module operable with the one or more radio frequency transceivers; and
one or more monitoring devices, each of the one or more monitoring devices comprising:
a mouth guard;
a biometric sensor; and
a communication module, operable with the biometric sensor and comprising:
an antenna;
a controller; and
a switch, responsive to the controller, and operative to change a radar cross section of the antenna by selectively altering a load impedance of the antenna;
wherein the one or more radio frequency transceivers are each configured to transmit a radio frequency signal and receive a backscattered return signal from the each of the one or more monitoring devices, each backscattered return signal having an output from the biometric sensor modulated therein due to the controller switching the switch.

16. The biometric monitoring system of claim 15, wherein the monitoring module is configured to collect the output from the biometric sensor and present indicia corresponding thereto on a display.

17. The biometric monitoring system of claim 16, wherein the indicia comprises data corresponding to an impact event detected by the biometric sensor.

18. The biometric monitoring system of claim 17, wherein the indicia further comprises a concussion warning.

19. The biometric monitoring system of claim 15, wherein the one or more radio frequency transceivers comprises a plurality of transceivers, further comprising a location determination module operable with the plurality of transceivers, wherein the location determination module is configured to determine a location of each of the one or more monitoring devices by determining a phase shift between the radio frequency signal and the backscattered return signal.

20. A method of monitoring anatomical motion, comprising:
providing a user with a mouth guard comprising a biometric sensor and a switch operable to change a radar cross section of an antenna of the mouth guard by selectively altering a load impedance of the antenna; and
monitoring biometric activity of the user by transmitting, with one or more transceivers, a radio frequency signal and receiving backscattered return signals having output of the biometric sensor encoded therein.

21. An electronic monitoring device, comprising:
a mouth guard;
a biometric sensor attached to or integrated with the mouth guard; and
a communication module, operable with the biometric sensor;
wherein the mouth guard defines a channel configured to receive a user's lips, wherein the biometric sensor is disposed on a first side of the channel and the communication module is disposed on a second side of the channel.
